(19) United States
(12) Patent Application Publication
(10) Pub. No.: US 2010/0224479 A1
(43) Pub. Date: Sep. 9, 2010

(54) MORPHOLOGY ENGINEERING OF AGGREGATES

(75) Inventors: Hans Moosmuller, Reno, NV (US); Rajan K. Chakrabarty, Reno, NV (US)

Correspondence Address: UNR/DRI Technology Transfer Office, UNR-DRI Technology Transfer Office, Mail Stop 321, Reno, NV, 89557, US

(73) Assignee: The Board of Regents of the Nevada System of Higher Educ., on Behalf of the Desert Res. Inst.

(21) Appl. No.: 12/698,954

(22) Filed: Feb. 2, 2010

Related U.S. Application Data
(60) Provisional application No. 61/149,109, filed on Feb. 2, 2009.

(51) Int. Cl. B01J 19/08; C07C 1/00

(52) U.S. Cl. 204/157.15; 422/186.04

(57) ABSTRACT

In various embodiments, the present disclosure provides, among other things, a system and method for influencing the morphology of aggregates. The present disclosure also provides for aggregates formed using the disclosed system and method. According to one disclosed method, a plurality of monomers are provided and an electric field is applied proximate the monomers. The applied fields helps influence the shape of an aggregate formed from the monomers.

### CROSS REFERENCE TO RELATED APPLICATION

This application claims the benefit of, and incorporates by reference, U.S. Provisional Patent Application No. 61/149,109, filed Feb. 2, 2009.

### STATEMENT OF GOVERNMENT SUPPORT

This invention was made with United States Government support under grants from the U.S. Department of Energy Atmospheric Science Program, Grant No. DE-FG02-05ER64008, and the National Science Foundation, Grant No. 0447416. The United States Government has certain rights in the invention.

### FIELD

The present disclosure relates, generally, to a method and system for engineering the morphology of aggregates, such as aggregates produced by flame synthesis. In some examples, particle morphology is engineered by one or both of applying an electric field during aggregate synthesis and controlling the temperature at which the aggregates are formed.

### BACKGROUND

Morphology can be an important property in particle-related applications. For example, morphology may be a factor in aerosol synthesis, which can be used for bulk production of nanomaterials, such as in—1) pharmaceuticals synthesis and processing, where the ability to control the size and state of agglomerates can influence their behavior in the human body; 2) synthesis of printer toners, tires, paints, fillers, and fiber-optics products, where uniformity of nanopowder morphology can influence product quality; and 3) carbon nanotube manufacturing, where uniformly sized and shaped carbon nanotubes may have desirable properties.

When used to produce particles beyond a certain length, aerosol formation mechanisms can produce agglomerates. Agglomerates can have complex, fractal-like morphologies, and particles having the same mass, such as being composed of the same number of individual particles, can have different morphologies, such as being more spherically or more linearly shaped.

Current methods of agglomerate production, such as flame synthesis, typically produce a mixture of morphologies. In addition, it is generally believed that such methods tend to produce agglomerates with mass fractal dimension of ≈1.8 in the cluster-dilute regime, and with mass fractal dimension of ≈2.4 in the cluster-dense regime.

### SUMMARY

The present disclosure provides a method for creating agglomerates in a manner that allows for tuning the morphologies, such as the morphology distribution, of the agglomerates. In various embodiments, the method involves one or more of applying an electric field during aggregate formation, controlling the temperature during particle formation, applying a shear field during particle formation, or adjusting the background medium used in particle formation.

When an applied electric field is used to influence aggregate morphology, a suitable electric field can be applied proximate the aggregate generating apparatus. The electric field may be applied, for example, by placing charged plates proximate the apparatus. However, any suitable method of applying an electric field may be used. In some embodiments, the applied field has an intensity of between about 1 μV/cm and about 10,000 V/cm, such as between about 1 V/cm and about 1000 V/cm, or between about 10 V/cm and about 500 V/cm. In a specific embodiment, the electric field is applied to produce aggregates having a more linear shape, that is, having a mass fractal dimension closer to 1. Increasing the field can produce aggregates having increasingly low fractal dimension.

When temperature is used to influence aggregate morphology, the temperature can be adjusted in a variety of ways. For example, the temperature can be adjusted by lowering the temperature of a combustion flame, such as by increasing the fuel-to-air equivalence ratio of a premixed flame or by changing amount or molecular/atomic weight of a gas, such as an inert gas, introduced into a flame. In one example, the temperature is adjusted by varying the amount of nitrogen in an air mixture used for combustion. In another example, an inert gas, such as SF₆, is added to the combustion atmosphere. The temperature can also be adjusted by heating or cooling the apparatus where particle aggregation takes place. In another example, explosive combustion followed by rapid quenching is used to lower the combustion temperature. Typically, cooler temperatures during aggregate formation will produce more linear aggregates/aggregates having a mass fractal dimension closer to 1. Cooler temperatures can enhance the effect of an electric field, including an applied electric field, present during aggregate synthesis, which can, in some examples, allow lower electric field strengths to be used.

When shear is used to influence aggregate size or shape, depending on the formation conditions and the level of shear applied, different results can be obtained. For example, lower shear levels can produce larger aggregates and/or aggregates having a more ball-like (less linear) structure. Higher shear levels can be used to cause fragmentation of the aggregate, leading to smaller aggregates.

Aggregate size/shape can also be influenced by the medium in which the aggregates are formed. For example, including an inert gas in the medium can result in larger particle formation compared with a strictly air medium. In some examples, helium gas can be used to produce larger particles and nitrogen gas can be used to produce smaller particles. The nature of the medium can also affect particle shape, with a helium-containing medium producing more highly spherical particles than a nitrogen-containing medium. In some examples, the particle shape or size is influenced by the thermal conductivity or molecular mass of the medium.

After aggregate formation, the aggregates can be further treated. For example, the aggregate production process may result in a population of aggregates having a distribution of morphologies, sizes, or other characteristics. Accordingly, one further treatment can be size selection, to select a certain size, size range, or size distribution of aggregates. In one implementation, the aggregate distribution is passed through an impactor.

When the population of aggregates has a distribution of morphologies, the population can be treated to select a certain morphology or certain morphological subpopulation or distribution of the aggregates. In one implementation, the particles are separated based on their flow properties, such as by using electrostatic classifiers to separate different morphologies based on their charge.

The present disclosure also provides an apparatus for carrying out the disclosed method. In one implementation, the apparatus includes an aggregate production unit, such as a flame generator, and an electric field generator, such as a pair of plates attached to a voltage source. In another implementation, the apparatus includes an aggregate production unit, such as a flame generator, and a temperature control unit, such as a refrigeration or heating unit, a heat sink, a gas source, or a flame stop. Either implementation may include additional components, including size selection components, such as an impactor, or morphology selection components, such as electrostatic classifiers.

There are additional features and advantages of the subject matter described herein. They will become apparent as this specification proceeds.

In this regard, it is to be understood that this is a brief summary of varying aspects of the subject matter described herein. The various features described in this section and below for various embodiments may be used in combination or separately. Any particular embodiment need not provide all features noted above, nor solve all problems or address all issues in the prior art noted above.

### BRIEF DESCRIPTION OF THE DRAWINGS

FIG. 1 is a schematic diagram illustrating one embodiment of a process for separating particles having different morphologies.

FIG. 2 is a flow chart of an aggregate formation method according to an embodiment of the present disclosure.

FIG. 3 is a schematic illustration of an apparatus for generating aggregates according to an embodiment of the present disclosure.

FIG. 4 is a schematic illustration of an apparatus for generating aggregates according to an embodiment of the present disclosure.

FIG. 5 is a schematic illustration of an embodiment of a system for generating aggregates according to the present disclosure and subsequently subjecting them to size and morphology selection.

FIG. 6 is graphs of the aggregate 3-d monomer number, N, versus the square root of the aggregate geometric mean diameter, LW, for aggregates formed according to an embodiment of the disclosed method at fuel-to-air equivalence ratios of 2.3 (a), 2.8 (b), and 3.5 (c).

FIG. 7 is scanning electron microscope images of a soot aggregate formed by typical methods (a) and soot particles produced according to an embodiment of the disclosed method at fuel-to-air equivalence ratios of 3.5 (b), 2.3 (c), and 5 (d).

### DETAILED DESCRIPTION

Unless otherwise explained, all technical and scientific terms used herein have the same meaning as commonly understood by one of ordinary skill in the art to which this disclosure belongs. In case of any such conflict, or a conflict between the present disclosure and any document referred to herein, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together. All numerical ranges given herein include all values, including end values (unless specifically excluded) and intermediate ranges.

Although methods and materials similar or equivalent to those described herein can be used in the practice or testing of the present disclosure, suitable methods and materials are described herein. The disclosed materials, methods, and examples are illustrative only and not intended to be limiting.

### DEFINITIONS

“Aerosol” refers to a dispersion of particles in a fluid medium, such as a gas, or in a vacuum. In some examples, the gas is air. Aerosols may be formed by a variety of methods, including ablation, flame synthesis, spray drying of colloidal or precipitated particles, spray pyrolysis, and thermal evaporation. The particle concentration in the aerosol is typically selected to provide suitable aggregate formation. In some examples the particle concentration is between about 10¹ particles/cm³ and about 10¹³ particles/cm³, such as between about 10⁴ particles/cm³ and about 10¹¹ particles/cm³ or between about 10⁷ particles/cm³ and about 10⁹ particles/cm³.

“Flow properties” refers to the influence of electrostatic and/or electrodynamic forces by themselves or in combination with other forces such as inertial, viscous, magnetic, or gravitational forces, optionally with the use of spatial or temporal gates, on a particle's movement in a fluid medium or a vacuum. In one example, the flow property is electrical mobility. According to the present disclosure, flow properties can be influenced by external forces in order to separate particles based on their morphologies.

In one example, time-of-flight techniques, such as time-of-flight mass spectrometry, are used to separate particles based on electrodynamic forces. In another example, an electrostatic classifier is used to influence the flow properties of a particle using viscous and electrostatic forces. A Millikan apparatus, using gravity and electrostatic forces, can be used to separate particles.

“Mobility diameter,” or Dₘ, is a parameter used to characterize particles and refers to the diameter of an equivalent sphere having the same electrical mobility as the particle in question, which may be nonspherical.

“Nanostructure” refers to a solid structure having a cross sectional diameter of between about 0.5 nm to about 500 nm. Nanostructures may be made from a variety of materials, such as carbon, silicon, and metals, including, without limitation, titanium, zirconium, aluminum, cerium, yttrium, neodymium, iron, antimony, silver, lithium, strontium, barium, ruthenium, tungsten, nickel, tin, zinc, tantalum, molybdenum, chromium, and compounds and mixtures thereof. Suitable materials that also are within the definition of nanostructures include transition metal chalcogenides or oxides, including mixed metal and/or mixed chalcogenide and/or mixed oxide compounds or carbonaceous compounds, including elemental carbon, organic carbon, and fullerenes, such as buckyballs, and related structures. In particular examples, the nanostructure is made from one or more of zinc oxide, silica, titanium dioxide, gallium nitride, indium oxide, tin dioxide, magnesium oxide, tungsten trioxide, and nickel oxide.

The nanostructure can be formed in a variety of shapes. In one implementation, the nanostructures are wires, such as wires having at least one cross sectional dimension less than about 500 nm, such as between about 0.5 nm and about 200 nm. Nanowires can be nanorods, having a solid core, or nanotubes having a hollow core. In some implementations, the cross sectional dimension of the nanostructure is relatively constant. However, the cross sectional dimension of the nanostructure can vary in other implementations, such as rods or tubes having a taper.

As used herein, “particle” refers to a small piece or quantity of an element, compound, or other material in the liquid or solid phase, or a mixture thereof. Particles that may be used in the present disclosure include those having a size, such as a cross-sectional diameter, of between about 1 nm and about 500 μm, such as between about 50 nm and about 100 μm, between about 50 nm and about 1 μm, or between about 200 nm and about 700 nm.

The shape or morphology of particles, including aggregates thereof, can be expressed in terms of the volume-to-surface area ratio, or density fractal dimension. Typical particles have a fractal dimension of between about 1 and about 3, typically greater than 1 and less than 2, such as between about 1.2 and about 1.8. The particles may be of any form, such as powders, granules, pellets, strands, or flocculent materials. The particles may be individual, discrete units, agglomerations or aggregates of multiple units, or mixtures thereof. Particle agglomerates can assume a number of shapes. As the number of particles in the agglomerate increases, the number of potential morphologies also typically increases. Even relatively small agglomerates typically can exist in a number of different morphologies.

The particles may be of any desired material that can be charged for a synthesis step or subsequent separation process, or that is mixed with such a material. For example, the particles may be, or include, carbonaceous materials, ceramics (such as metal borides, carbides, or nitrides), extenders, fillers, inorganic salts, metals, metal alloys, metal alkoxides, metal oxides, pigments, polymers, zeolites, or combinations and mixtures thereof. Specific examples of such materials include aluminum, antimony oxide, asbestos, attapulgite, barium sulfate, boehmite, calcium carbonate, chalk, carbon black, chromium, cobalt, copper, diatomaceous earth, fumed oxides, gold, halloysite, iron, iron oxides, kaolin, molybdenum, montmorillonite, nickel, niobium, palladium, platinum, silica, silica aerogels, silica sols, silicon, silver, tantalum, titania, titanium, titanium isopropoxide, zinc oxide, zinc sulfide, or alloys, mixtures, or combinations thereof.

In some examples, the particles can be used to form nanostructures, such as nanorods or nanotubes. The disclosed system and method can be used to synthesize and separate aggregates, such as nanoparticles, having different morphologies and, optionally, other differences. In some examples, the particles are combustion particles, such as carbon black.

One particle property is electric charge. At a given temperature, particles typically have a Boltzmann distribution of positively charged, negatively charged, and neutral particles. Particles may be further charged by various mechanisms, including static electrification, such as electrolytic charging, spray electrification, or contact charging. Field charging may also be used. Other forms of charging include corona discharge, radioactive discharge, ultraviolet radiation, and flame ionization. In some implementations, a combination of charging mechanisms is used.

In a specific example, bipolar charging is used. In bipolar charging, positive and negative charges are applied to the particles, such as by interacting the particles with bipolar ions. The net charge on the particles following bipolar charging may be negative, positive, or neutral. When charged, the particles may have at least one charge, but potentially may be multiply charged.

The distribution of charges is typically related to temperature. In some examples, a sample is cooled prior to charging in order to produce a desired distribution. For example, higher temperatures may facilitate multiple morphologies being multiply-charged, which may make separation less efficient. Cooling the sample can increase the probability that the more linear morphologies are multiply-charged rather than more spherical morphologies.

In some embodiments, the particles are at least partially charge deformable. “Charge deformable” means that the particles change shape, such as elongating, when charged, such as with an electrostatic charge. Charge deformable particles are typically at least partially flexible. In some examples, particles elongate when a charge, or charges, is applied or increased. In another example, particles assume a more spherical shape when charge is removed or reduced.

In further embodiments, when an aggregate is built from smaller monomers or other subunits, the growing aggregate has a dipole moment. The aggregate being created experiences an electric field, which may be present as part of a combustion unit, such as a flame in a flame synthesis configuration, or an externally applied field. The electric field orients the growing aggregate and aligns the monomers or subunits such that the additional monomers are added in an altered fashion. For example, a suitable electric field, or suitable combination of temperature and electric field, can result in aggregates having a more linear morphology, such as evidenced by a lower (closer to 1) mass fractal dimension.

Morphology of particles is also related to mobility. The following equation provides a relationship between mobility and particle size:

$${Z}_{p} = \frac{{ieC}_{c}}{3 πμ {D}_{p}} \qquad ( 1 )$$

In Equation 1, e is the elementary charge, i is the number of elementary charges carried by a particle (typically an integer), C_{c} is the slip correction factor (defined later in this disclosure), and μ is the gas viscosity. As can be seen from this equation, for a given mobility Zₚ, more highly charged particles will have a larger Dₚ compared with less charged particles.

In various embodiments of the present disclosure, particles with a certain charge can be selected using forces that depend on the charge, such as electrostatic and/or electrodynamic forces by themselves or in combination with other forces such as inertial, viscous, or gravitational forces or with the use of spatial or temporal gates. In a specific example presented in the present disclosure, particles with a certain charge are selected using a differential mobility analyzer. The differential mobility analyzer uses a combination of viscous and electrostatic forces to select a combination of charge q and aerodynamic particle size with a spatial gate.

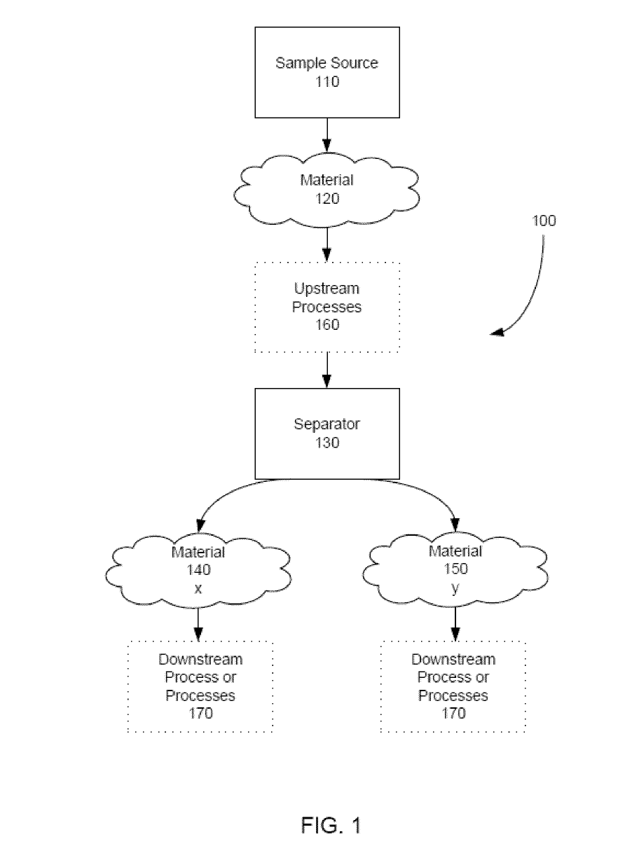

A schematic diagram of a process for separating particles based on flow properties 100 is presented in FIG. 1. A sample source 110 produces a material 120 having a plurality of particles, at least a portion of which differ at least in their morphology, although the particles of the portion may differ in other properties, such as size.

The material 120 is typically dispersed in a carrier fluid, such as a gas. In one example, the gas is air. The concentration of the material 120 in the carrier gas may depend on a number of factors, such as the type of separator used in the process 100, the flow rate of the material 120 through the separator, the properties of the material 120, such as its size, size distribution, morphology, morphology distribution, or other chemical or physical properties. For example, in at least some processes it may be desirable to keep the concentration of the material 120 below a certain level to avoid undesired particle agglomeration.

A variety of methods are known to prepare suitable concentrations of particles 120 in a carrier. For example, particles, such as carbon black particles, can be produced by flame combustion. Ablation of solid materials is another technique that can be used to generate the particles 120.

The material 120 is passed through a separator 130 that separates the material 120, at least in part, based on morphology. Suitable separators 130 include those that can separate materials based on their flow properties, such as their electrical mobility. One suitable separator 130 that can separate particles on their electrical mobility is the differential mobility analyzer. Suitable differential mobility analyzers include the Series 3080 electrostatic classifiers, available from TSI, Inc. of Shoreview, Minn. Details of an instrument that allows relatively high particle concentrations to be used are described in Camata et al., “Deposition of Nanostructured Thin Film from Size-Classified Nanoparticles,” NASA 5th Conference on Aerospace Materials, Processes, and Environmental Technology (November 2003), incorporated by reference herein to the extent not inconsistent with the present disclosure. In some aspects, the process 100 separates the material 120 only on the basis of morphology. In other aspects, the process 100 separates the material 120 based on morphology and at least one other property, such as size or charge.

As shown in FIG. 1, the separator 130 separates the material 120 into two subsets, one set 140 has a charge of xe and another set 150 has a charge of ye. In at least some examples, x and y are integers and the overall net charge is xq or yq, where q is the elementary charge of 1.602176487×10⁻¹⁹ C. The separator 130 may separate the material into more than two subsets. Each subset may have particles having only a single charge, or single distribution of charges, or may have multiple charges or charge distributions. At least one subset may have a random charge distribution.

Although FIG. 1 illustrates a single separator 130, alternative embodiments of the system 100 may include multiple separators. When multiple separators 130 are used, the separators 130 may be the same or different. Multiple separators 130 may be desirable, for example, when the material 120 includes more than two morphologies or particle sizes that are desired to be separated. When multiple separators 130 are used, additional processes (not shown) may be performed on the material 120 after it exits the separator 130 and before it enters one or more additional separators. When multiple separators 130 are used, they may be present as discrete components or devices or may be a unitary device.

The system 100 optionally includes one or more upstream processes 160. One upstream process may consist of size selection. Suitable impactors and virtual impactors are commercially available, such as the Sioutas Cascade Impactor from SKC Inc., of Eighty Four, Pa. and the Model 3306 Impactor Inlet from TSI Inc., of Shoreview, Minn. Size selection may be useful, for example, in providing an initial separation of the material 120 to remove undesired components, such as particles outside a desired particle range. In one example, size selection is accomplished using an impactor or a virtual impactor.

Upstream processes 160, such as size selection, may be useful in reducing fouling of the separator 130 or in increasing separator 130 efficiency or producing more desirable subsets 140 or 150.

The system 100 optionally includes one or more downstream processes 170. Downstream processes 170 can include, but are not limited to, treatment, detection, or separation processes.

### DESCRIPTION

In various embodiments, the present disclosure provides an apparatus and a method for engineering the morphology of aggregates by one or more of applying an electric field during aggregate formation, controlling the temperature during aggregate formation, applying a shear field during aggregate formation, or controlling the medium in which aggregates are formed.

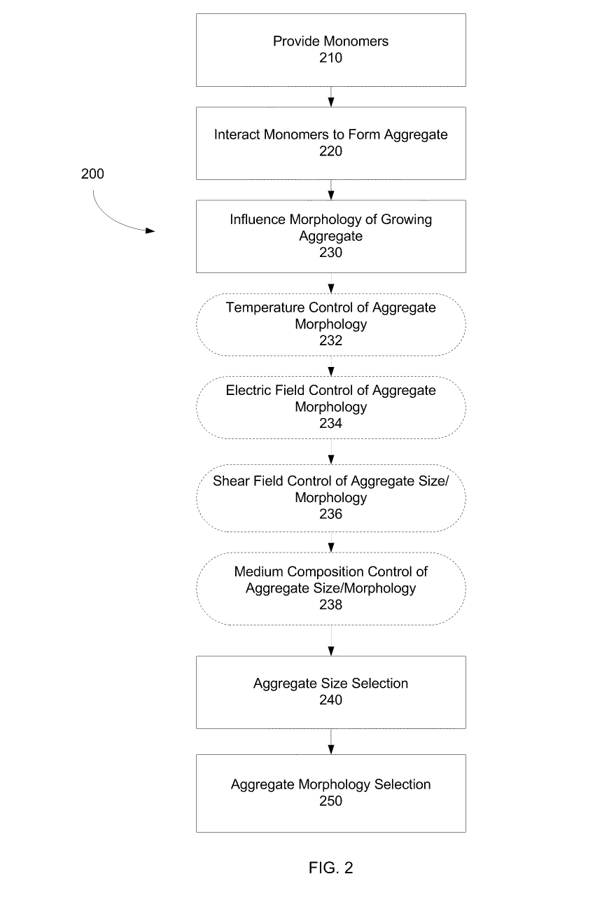

FIG. 2 is a flowchart of a method 200 of the present disclosure. In step 210, a plurality of monomers, such as particles, are provided. In step 220, the monomers are interacted to form an aggregate. In step 230, while the monomers are forming the aggregate, the morphology and/or size of the growing aggregate are influenced by one or more processes 232-238.

In process 232, particle morphology is influenced by controlling the temperature during aggregate formation. In process 234, an applied electric field or charge source is used to influence particle morphology. Shear fields are used to influence particle size/morphology in process 236. The composition of a medium in which aggregates are formed is used to influence particle size/morphology in process 238. Details of processes 232-238 are described further below.

After step 230, and any processes 232-238, the aggregates can optionally be separated from one another based on their size in step 240. In optional step 250, the aggregates can be separated based on their morphology. Suitable processes for size or morphology selection are described elsewhere in this disclosure.

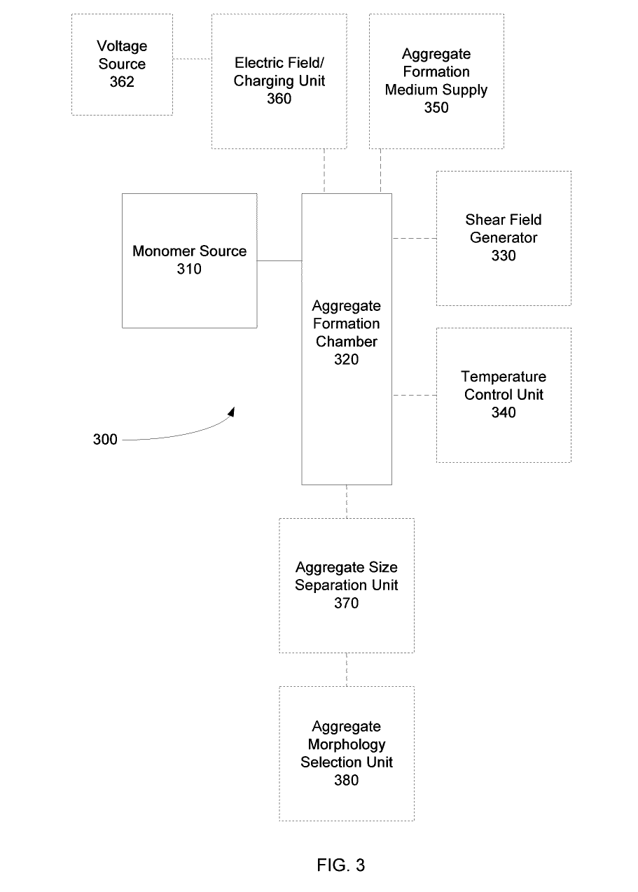

FIG. 3 illustrates an aggregate generation apparatus 300. The apparatus 300 includes a monomer source 310. In one example, the monomer source is a flame generator producing carbon black particles. Various monomer sources can be used depending on a number of factors, such as the desired aggregate morphology, aggregate size, and aggregate composition. The monomer source 310 is coupled to an aggregate formation chamber 320. In the aggregate formation chamber, the monomers come together to form the aggregate.

Consistent with method 200 of FIG. 2, the apparatus 300 optionally includes additional components depending on which processes (232-238) are used to influence aggregate size/morphology. When temperature control is used (process 232), the apparatus 300 includes a temperature control unit 340. Suitable temperature control methods and units are described elsewhere in this disclosure, but include heating or cooling units, quench gas sources, heat sinks, and gas flow controls.

When electric field or charges are used to influence particle morphology (process 234), the apparatus 300 includes an electric field/charging unit 360. One suitable charging unit 360 is plates located proximate, including inside, the aggregate formation chamber 320, and attached to a voltage source 362.

When shear fields are used to influence aggregate size/morphology (process 236), the apparatus 300 includes a shear field generator 330. Shear field generator 330 may be, for example, a gas outlet fluidly coupled to the aggregate formation chamber 320.

When the composition of a medium used in the aggregate formation chamber 320 is used to influence aggregate size/morphology, the apparatus 300 includes a medium supply 350. The medium supply 350 is configured to supply one or more medium elements, such as gasses, in the proper proportion to the aggregate formation chamber 320.

After aggregation formation, the aggregates can optionally be transmitted to an aggregate size selection unit 370 and/or an aggregate morphology selection unit 380. Suitable aggregate size selection units 370 and morphology selection units 380 are described elsewhere in this disclosure.

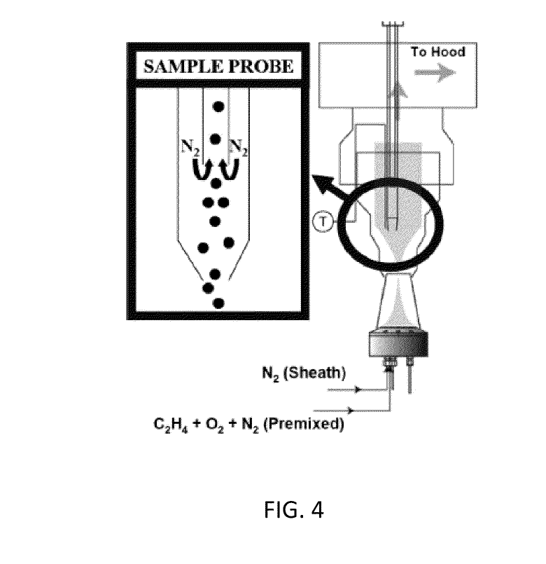

FIG. 4 illustrates an example of an apparatus useable to generate carbon aggregates. A premixed gas of ethene and oxygen diluted with nitrogen is passed through a 6 cm diameter porous frit. The frit is surrounded by a 0.5 cm wide annular sheath region through which nitrogen gas is passed. However, the dimensions of the apparatus can be varied as desired for a particular application. In addition, other aggregate generation systems can be used, including those not using a premixed gas, or those using other methods of forming aggregates from monomers or other subunits.

When an applied electric field is used to influence aggregate morphology, a suitable electric field can be applied proximate the aggregate generating apparatus, such as the apparatus of FIG. 3. The electric field may be applied, for example, by placing proximate the apparatus plates attached to a voltage source. However, any suitable method of applying an electric field may be used.

The nature of the electric field to be used for a particular application typically depends on a number of factors, including the nature of the material from which the aggregates will be formed, the synthesis method, the distance from the source of the field to the point where the aggregates are formed, the temperature during aggregate formation, and the particular morphology or morphology distribution desired. In some cases the electric field is static. In other cases the electric field is varied, including pulsed fields, ramped potential fields, and fields of alternating potential or polarity. In some embodiments, the applied field has an intensity of between about 1 μV/cm and about 10,000 V/cm, such as between about 1 V/cm and about 1000 V/cm, or between about 10 V/cm and about 500 V/cm. Typically, larger electric fields will produce more linear aggregates/aggregates having a mass fractal dimension closer to 1.

When temperature is used to influence aggregate morphology, the temperature can be adjusted in a variety of ways. For example, the temperature can be adjusted by lowering the temperature of a combustion flame, such as by increasing the fuel-to-air equivalence ratio of a premixed flame. The temperature can also be adjusted by heating or cooling the apparatus in which particle aggregation takes place. For example, the aggregation temperature for the particles can be significantly lowered by extracting the particles from a flame before they fully aggregate into a chamber maintained at room temperature (300 K). Note that a chamber being maintained at 300 K has its kT at 1/7^{th} than that of the flame (T ˜2100 k). In another example, cooler combustion is produced using explosive combustion followed by rapid quenching, such as using a solid heat sink or a flame trap. Typically, cooler temperatures during aggregate formation will produce more linear aggregates having a mass fractal dimension closer to 1. In addition, application of an external electric field to the chamber can further reduce the dominance of Brownian diffusion motion of the monomers and FAs inside of the chamber, and emphasize the effects of the electric forces. This can result in a larger fraction of near-linear, low mass fractal dimension of aggregates produced inside of the chamber. Cooler temperatures can enhance the effect of an electric field, including an applied electric field, present during aggregate synthesis, which can, in some examples, allow lower electric field strengths to be used.

Another technique that can be used to alter aggregate size/shape, including in conjunction with applied electric field, is the application of a shear field. Lower shear levels can be used to produce larger aggregates (having more particles) and/or aggregates having a higher fractal dimension. Higher shear levels can be used to cause aggregate fragmentation, which can lead to smaller aggregates. In some situations, higher shear can be used to produce particles having a higher fractal dimension.

Aggregate size/shape can also be influenced by the medium in which the aggregates are formed. For example, including an inert gas in the medium can result in larger particle formation compared with a strictly air medium. In some examples, helium gas can be used to produce larger particles and nitrogen gas can be used to produce smaller particles (than particles produced using helium). The nature of the medium can also affect particle shape, with a helium-containing medium producing more highly spherical particles than a nitrogen-containing medium. In some examples, the particle shape or size is influenced by the thermal conductivity or molecular mass of the medium.

After aggregate formation, the aggregates can be further treated. For example, the aggregate production process may result in a population of aggregates having a distribution of morphologies, sizes, or other characteristics. Accordingly, one further treatment can be size selection, to select a certain size, size range, or size distribution of aggregates. One suitable technique for size selection is passing the aggregate distribution through an impactor. One suitable technique for morphology selection is through the use of particle flow properties, such as using electrostatic classifiers or differential mobility analyzers to separate particles based on their charge, as described in U.S. patent application Ser. No. 12/165,511, incorporated by reference herein to the extent not inconsistent with the present disclosure.

The following Example is provided to illustrate specific features of one disclosed embodiment of the present disclosure. A person of ordinary skill in the art will understand that the scope of the present disclosure is not limited to these particular features.

### EXAMPLE

This Example describes ensembles of cluster-dilute soot fractal-like aggregates (FAs) with much lower mass fractal dimension (i.e., 1.2-1.5) than have typically been reported for agglomerates produced by flame synthesis techniques. The soot FAs were produced in the soot-formation window of a premixed ethene (C₂H₄)-oxygen (O₂) flame. An established charge-based particle segregation technique was used to segregate elongated FAs for morphology analysis with scanning electron microscopy (SEM) and image processing.

The ethene-oxygen premixed flame setup (FIG. 4) used for producing soot FAs has previously been described in detail. Chakrabarty et al., Applied Optics 46, 6990 (2007); Slowik et al., Aerosol Science and Technology 41, 295 (2007). The mass flow rates of ethene and oxygen were varied to operate the flame with four fuel-rich fuel-to-air equivalence ratios φ—2.3, 2.8, 3.5, and 5.0. For each equivalence ratio, soot FAs were sampled in the overfire region of the flame, where the characteristic flame residence times are roughly an order of magnitude longer than the laminar smoke point residence time. Soot particle monomer number concentration in the long residence time regime is independent of axial position, which facilitates sampling of a steady and uniform distribution of particles. Sampled soot particles were passed through an impactor to remove particles larger than ≈5 μm in diameter.

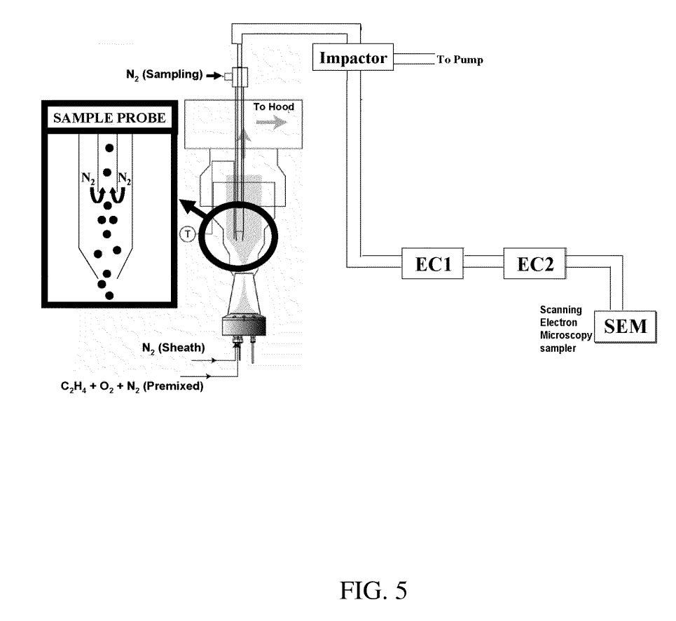

Particle flow exiting the impactor was directed through two identical Electrostatic Classifiers (ECs) in series (FIG. 5). The EC utilizes a combination of a viscous and electrostatic force to select a combination of net charge q and electrical mobility diameter Dₘ with a spatial gate. ECs are widely used for particle sizing and for the generation of monodisperse aerosols in the size range from 0.005 to 1.0 μm. The particles were bipolarly charged using a neutralizer (model Kr-85, TSI Inc., St. Paul, Minn., USA) before entering either of the ECs (Model 3080, TSI Inc., St. Paul, Minn., USA). The ECs were calibrated using National Institute of Standards and Technology (NIST) certified Polystyrene Sphere Latex (PSL) particle size standards.

A detailed description of the morphology segregation technique is provided in U.S. patent application Ser. No. 12/165,511, incorporated by reference herein to the extent not inconsistent with the present disclosure. However, the following general discussion is provided. This segregation technique is based on the discovery that more elongated particles are more likely to become doubly charged than more compact particles of the same mass because it requires less energy to add the second charge at a larger distance from the first one. The sheath flow-rate of the 1^{st} EC was set at around 8 l/min STP to select singly charged (i.e., q=−e) particles with electrical mobility diameter Dₘ≈280 nm and doubly charged (i.e., q=−2e) particles with Dₘ=460 nm. The particles exiting the 1^{st} EC were neutralized and sent to the 2^{nd} EC, which was set to select singly charged particles with Dₘ=460 nm. The particles selected by the 2^{nd} EC are the same 460 nm particles that were originally selected by the 1^{st} EC with a net double charge on them. This technique of shape selecting particles typically is more effective if the sample contains enough particles, with a morphology facilitating the placement of double charges, in the desired size range. In samples containing particles with near-alike morphologies, this charge-based morphology separation technique may be more difficult to apply or provide less effective results than for more optimal conditions.

For example, unsuccessful attempts were made to segregate morphologies of FAs with Dₘ<200 nm from the premixed flame reported in this Example. The majority of these doubly charged, small FAs (Dₘ<200 nm) had mass fractal dimensions of ≈1.8, typical of 3-D FAs. These small FAs consist of only few monomers resulting in a more limited number of possible shapes. Unlike FAs with a large number of monomers, small FAs have a more spherical morphology with little opportunity for segregating more spherical from more elongated aggregates. Therefore, the particle number concentration exiting the 2^{nd} EC was a very small fraction of the sample population.

In this Example, the combination of two ECs segregated more elongated FAs, constituting ≈3% of the total sub-micron sized particles sampled from the flame. The segregated particle size distribution from the EC is Gaussian with a peak of Dₘ≈460 nm. Soot particles exiting the 2^{nd} EC were impacted with a flow rate of 2(1 min⁻¹) STP onto 10-μm thick nuclepore clear polycarbonate filters for SEM analysis. It has been observed during past studies that the original particle structure of impaction-collected FAs is modified only very minimally upon impaction onto the filter at the pump-suction flow rate used in this study. After sampling, the filter samples were kept in refrigerated storage and later prepared for SEM analysis by coating them with a 1-nm thick layer of platinum to prevent aerosol charging during SEM analysis. The coated filters were analyzed using a Hitachi Scanning Electron Microscope (Model S-4700).

For each fuel-to-air equivalence ratio, images of ≈150 individual doubly charged particles were analyzed for their mass fractal dimension. For a 3-d fractal aggregate in the cluster-dilute regime, parts of the aggregate can randomly screen other parts during 2-d imaging. After correcting for this screening, the projected 2-d fractal dimension of FAs approximately equals their actual 3-d fractal dimension for 3-d fractal dimensions smaller than two. The 3-d monomer number N in eq. 1 can be calculated from the 2-d projected image using the equation:

$$N = {( \frac{{A}_{agg}}{{A}_{mon}} )}^{κ} \qquad ( 2 )$$

where κ=1.10 corrects for the 2-d screening effect, A_{agg} is the 2-d aggregate projected area, and Aₘₒₙ is the mean projected monomer area. The FA properties quantified using image analysis include A_{agg}, Aₘₒₙ, L (projected aggregate length), and W (aggregate projected width normal to L). The parameter (LW)^{0.5}—defined as the geometric mean diameter of an aggregate—has been shown to be a good approximate for the radius of gyration and eq. 1 can be re-written as:

N˜[\sqrt{LW}]^{D}^{_{f}} (3)

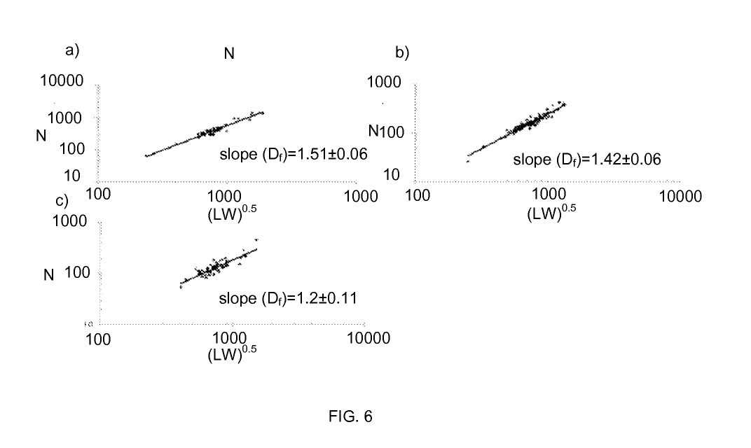

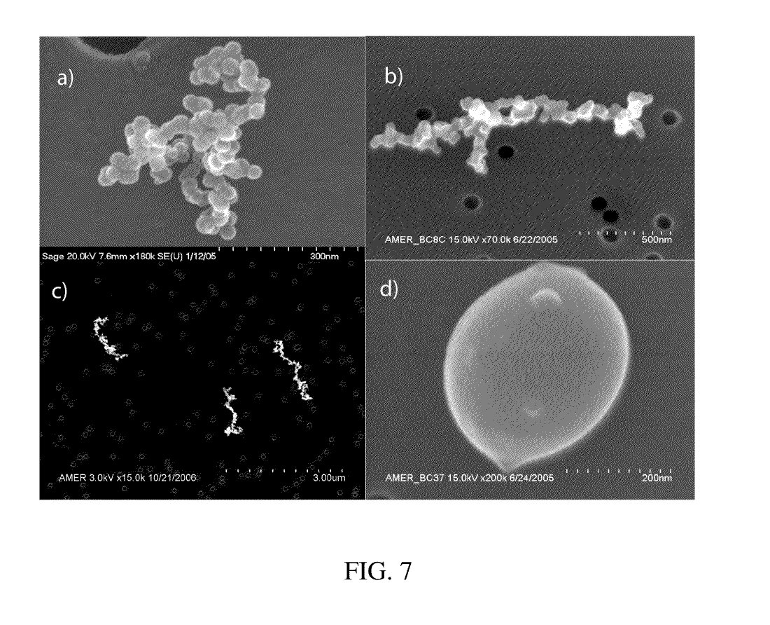

FIG. 6 shows N vs. (LW)^{0.5} data plotted on a log-log scale for the doubly charged soot FAs with an electrical mobility diameter Dₘ=460 nm and for three fuel-to-air equivalence ratios (i.e., 2.3, 2.8, and 3.5). On the log-log scale, there is a clear linear relationship between N and (LW)^{0.5}. The slope of this relationship is the mass fractal dimension D_{f}, which is determined with a linear least-square fit. As the fuel-to-air equivalence ratio is increased from 2.3 to 3.5, the mass fractal dimension D_{f} monotonically decreases from 1.51 to 1.20, upon further increase of the fuel-to-air equivalence ratio to 5.0, the particles become near-spherical with a mass fractal dimension of ≈3 (FIG. 7d) due to condensation of organics emitted by the flame. These results contains two novel features, i) low mass fractal dimensions much smaller than the typical 1.8 are, for the first time, observed in a premixed flame and ii) the fractal dimension is a function of fuel-to-air equivalence ratio, monotonically decreasing with increasing fuel-to-air equivalence ratio in the range of 2.3 to 3.5.

Most simulation studies have investigated aggregation of neutrally charged particle populations due to Brownian motion, and have found that the fractal dimension of the resulting aggregates had a narrow distribution centered around 1.8 in the asymptotic, equilibrium limit. These findings support that the observed low fractal dimensions in this Example are not statistical outliers of a broad distribution of fractal dimensions. The observation of mass fractal dimensions much smaller than the 1.8 typical for 3-d DLCA can possibly be explained by two different mechanisms—1) some growth in a premixed flame occurs via 2-d DLCA mechanism, and 2) the electric field inside a flame causes partial alignment of aggregates with a dipole moment and non-Brownian diffusion of charged monomers during the aggregation process.

Past simulation studies observed 2-d DLCA cluster-dilute aggregation to generate FAs with mean fractal dimensions between 1.40 and 1.44. Sorensen et al. reported in-situ observation of large 2-d FAs produced in the annular region of a diffusion flame, whose flame properties are quite different from the premixed flame used here. Langmuir 17, 5431 (2001). Visual inspection of the FA images obtained in this Example (FIGS. 7b, c) shows the aggregates to be unusually elongated and possibly flat; i.e., one of their three dimensions is significantly smaller than the other two. Based on the reasoning by Sorensen et al., it is believed that 2-d growth of FAs could possibly have taken place in the annular region of the premixed flame used in this Example. The thin, annular region may constrain the radial dimension of FAs to much smaller values than either their azimuthal or axial dimensions.

The second possibility is of an electric field in the flame orienting aggregates and aligning the movement of monomers during the aggregation process, thereby reducing its dimensionality. While aggregation of particles has been studied extensively with numerical simulations, very few studies have examined the effects of particle charges, dipole moments, and electrical fields on aggregation. The ionic/charged environment of a flame can give rise to a distribution of monomer and aggregate charge due to the random nature of the charging process. Such a charge distribution can increase the rate of aggregation above that of neutral particles and aggregates. Large FAs, in flames, often have a dipole moment that tends to align these aggregates parallel to electric field lines, which is parallel to the preferential direction of movement for charged monomers. If electric forces were dominant and all aggregates had dipole moments and all monomers were charged, one would expect near-linear shaped FAs (i.e., linear chains with D_{f}≈1) to be formed. However, in a flame environment not all particles are necessarily charged or have dipole moments and the effect of alignment of particles and their movement competes with Brownian motion. This may result in a majority population of aggregates formed through 3-d DLCA with a mass fractal dimension of ≈1.8 (FIG. 7a) and a minority population with a lower mass fractal dimension as observed in this Example. It was hypothesized that the competition of random Brownian motion with alignment due to electrostatic forces may lead to more chainlike aggregates with a lower fractal dimension at lower aggregation temperatures. This is exactly what is established in this Example. Increasing the fuel-to-air equivalency ratio of the premixed flame used in this Example lowers the flame temperature and thereby lowers the mass fractal dimension as observed (FIG. 6).

It was observed that at extremely fuel-rich conditions (i.e., φ≈5), particles produced by the flame had near-spherical shapes (i.e., D_{f}≈3; see FIG. 7d). Previous studies have observed a similar phenomenon, finding that as the fuel-to-air equivalency ratio increases to above 4.0 in a premixed flame, a sharp transition in particle properties occurs including a drastic increase of the ratio of polycyclic aromatic hydrocarbons (PAHs) to black carbon (BC) and an increase of the mass fractal dimension from 1.7±0.15 to 2.95±0.10. One explanation offered for this observation was that as the concentration of PAHs in a flame increases beyond a certain limit, the pores and irregularities of the FA get filled by the condensed PAH compounds and the individual monomers lose their identity. At this transition point the FAs become nearly smooth spheres.

In summary, a small population (≈3%) of sub-micron-sized (≈460 nm) soot FAs produced in the soot formation window of a premixed ethene-oxygen flame were segregated. It was observed that their mass fractal dimension was unusually low (i.e., between 1.2 and 1.5). Their formation mechanism may have been influenced by either a 2-d DLCA growth mechanism and/or by electrostatic forces inside of a flame, thereby explaining their extremely low mass fractal dimension. The electrostatic force hypothesis could also explain the observed decrease of fractal dimension with the increasing fuel-to-air equivalence ratio of the flame.

The use of an applied electrostatic field can be used to control the fractal dimension and associated properties of soot.

It is to be understood that the above discussion provides a detailed description of various embodiments. The above descriptions will enable those of ordinary skill in the art to make and use the disclosed embodiments, and to make departures from the particular examples described above to provide embodiments of the methods and apparatuses constructed in accordance with the present disclosure. The embodiments are illustrative, and not intended to limit the scope of the present disclosure. The scope of the present disclosure is rather to be determined by the scope of the claims as issued and equivalents thereto.

We claim:

1. A method for forming aggregates:
providing a plurality of monomers; and
applying an electric field proximate the monomers;
whereby the monomers form an aggregate and the applied electric field orients a building aggregate and aligns the movement of the monomers relative to the orientation of the building aggregate.

2. The method of claim 1, wherein providing a plurality of monomers comprises combusting a fuel source.

3. The method of claim 2, wherein the fuel source comprises ethene and oxygen.

4. The method of claim 1, further comprising altering the temperature proximate the monomers.

5. The method of claim 4, wherein altering the temperature proximate the monomers comprising lowering the temperature to produce more linear aggregates or aggregates having a lower fractal dimension.

6. The method of claim 2, wherein the fuel source comprises a combustible material and oxygen, and altering the temperature proximate the monomers comprises diluting the fuel to decrease the fuel-to-air equivalence ratio.

7. The method of claim 6, wherein the fuel combustible material comprises ethene.

8. The method of claim 1, wherein the electric field is applied at a ramped potential.

9. The method of claim 1, wherein the electric field is applied in a pulsed manner.

10. The method of claim 1, wherein the electric field has an alternating polarity.

11. The method of claim 1, wherein the electric field has an alternating potential.

12. The method of claim 1, wherein applying an electric field proximate the monomers is carried out to produce more linear aggregates.

13. The method of claim 1, wherein applying an electric field proximate the monomers is carried out to produce aggregates having a lower fractal dimension.

14. An aggregate generation apparatus comprising:
an aggregate generation unit; and
an electric field generator proximate the aggregate generation unit.

15. The aggregate generation apparatus of claim 14, further comprising a size selector coupled to an output of the aggregate generation unit.

16. The aggregation generation apparatus of claim 14, further comprising a morphology selector coupled to an output of the aggregate generation unit.

17. The aggregate generation apparatus of claim 16, further comprising a temperature adjuster proximate the aggregate generation unit.
